(19) United States
(12) Plant Patent Application Publication
(10) Pub. No.: US 2019/0223339 P1
(43) Pub. Date: Jul. 18, 2019

(54) Raspberry plant - 'TITANIA' cultivar

(50) ;

(71) Applicant: SANT'ORSOLA SOCIETA' COOPERATIVA AGRICOLA, Pergine Valsugana (IT)

(72) Inventors: Sara Giacomelli, Vigolo Vattaro (IT); Marco Giacomelli, Vigolo Vattaro (IT); Alessandra Viliotti, Vigolo Vattaro (IT)

(21) Appl. No.: 16/350,826

(22) Filed: Jan. 18, 2019

(30) Foreign Application Priority Data
Jan. 18, 2018 (QZ) 20180035

(51) Int. Cl. A01H 6/74

(52) U.S. Cl. PLT/204

(57) ABSTRACT

A new a primocane raspberry cultivar named ‘Titania’ which produces berries which are of a very large size with a good colour and flavour is disclosed. The new cultivar has the advantages of producing two crops per year, wherein the fruits mainly ripen on current season's cane in autumn.

Genus and species: Rubus idaeus L.

Variety denomination: ‘Titania’.

### BACKGROUND OF THE NEW VARIETY

A new and distinct cultivar of red raspberry (Rubus idaeus L.) hereinafter referred to by the cultivar denomination ‘Titania’ is disclosed. The new variety ‘Titania’ was obtained from the controlled cross between the varieties Vajolet (mother plant) and the pollen of a wild mountain raspberry plant (father plant).

The crossing for the new cultivar occurred in year 2009 in Altopiano della Vigolana (Trento - Italy) and it was subjected to selection in 2010, and successively tested until 2017. The characteristics of the new cultivar have been found stable and have been transmitted without change through succeeding (more than 7 years) asexual propagations by root cutting.

Plant breeder's right were applied for with the European Union on Jan. 18, 2018. ‘Titania’ has not been made publicly available.

### SUMMARY OF THE INVENTION

The most outstanding and distinguishing characteristics of the ‘Titania’ cultivar are hereunder reported.
Primocane raspberry plant;
Very vigorous and healthy plant;
Very large fruit size;
Good fruit colour and flavour.

The new variety produces two crops per year and the fruits mainly ripen on current season's cane in autumn.

The new variety can be propagated by: the use of suckers produced in a spawn-bed, root cutting and use of tissue culture techniques. The root cutting is to be preferred.

### BRIEF DESCRIPTION OF THE PHOTOGRAPHS

The new red raspberry cultivar ‘Titania’ is illustrated by the accompanying photographs showing typical specimens of the new variety in colour as true as reasonably possible to make the same colour illustrations of this character by conventional photographic procedures.

FIG. 1 is a view showing the raspberry plant with growing fruits. The upright and tall plants with very vigorous lateral shoots, mainly present on the apical part of the plant, can be appreciated.

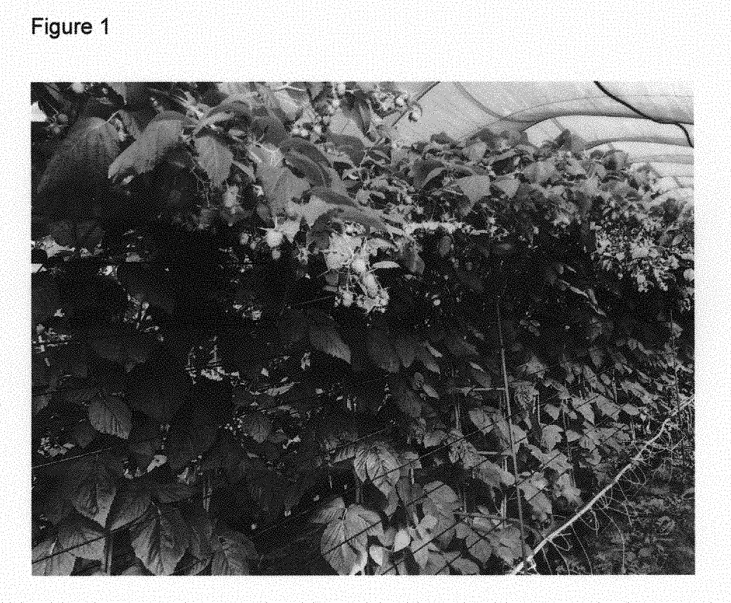

FIG. 2 is a view showing the leaf upper and lower surfaces.

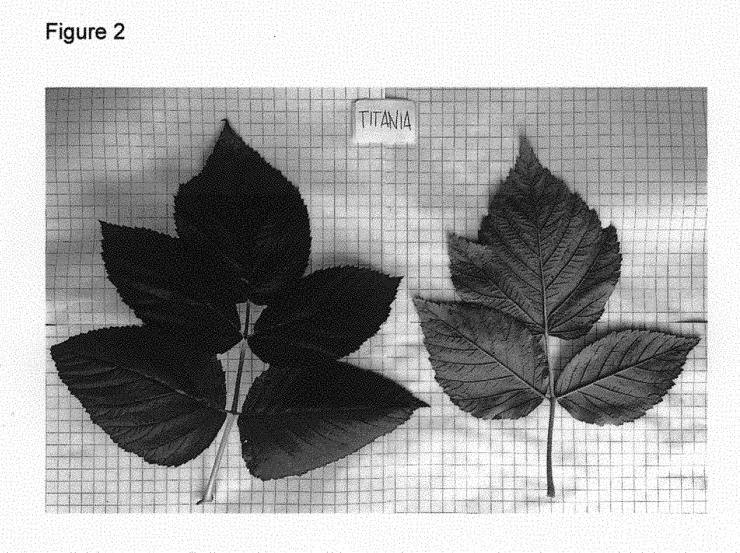

FIG. 3 is a view showing the plant primocane internode length, cane colour and thorns.

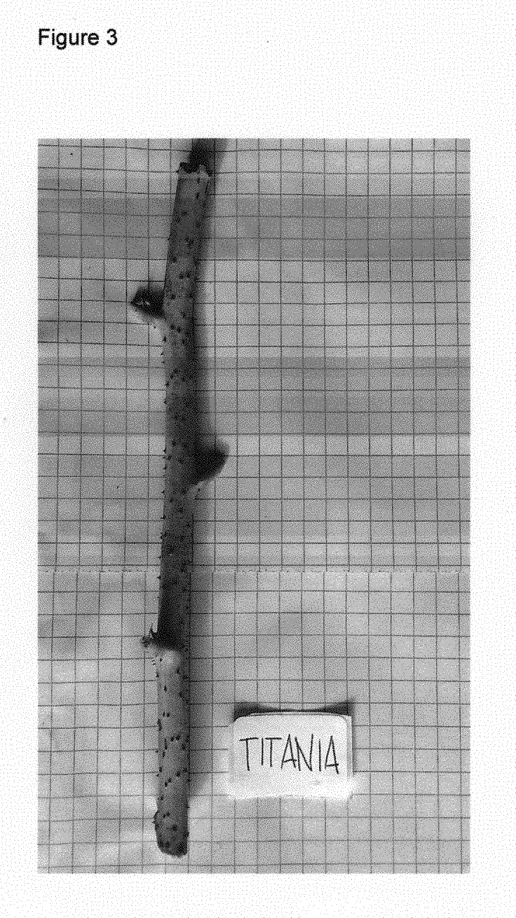

FIG. 4 is a view showing the ripe fruit size and shape.

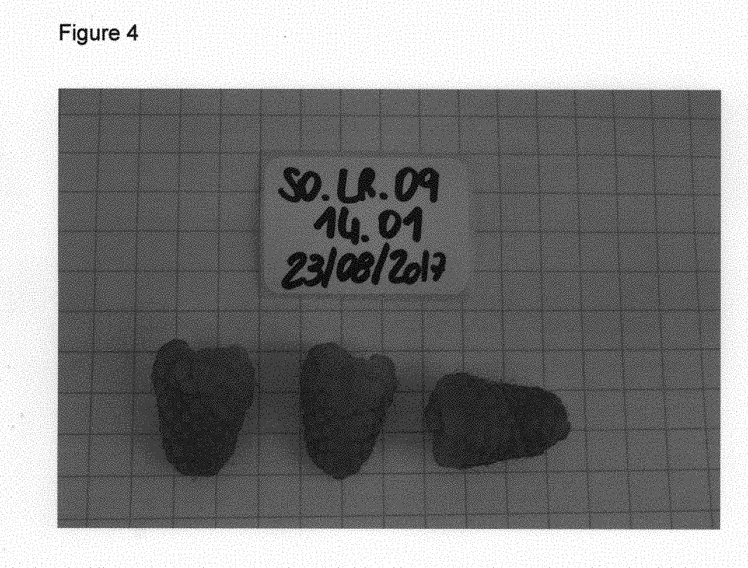

### DETAILED DESCRIPTION

The following detailed description set forth the distinctive characteristics of the new variety ‘Titania’. The performance of new cultivar for retention of its distinctive characteristics has been evaluated through successive asexual propagations by root cutting in the Applicant's setting in Trentino (Italy) under natural conditions in high tunnel. The growing was in soilless using coir as substrate.

The plant assessment was performed for 2 years.
Classification:
   Family.—Rosaceae.
   Botanical.—Rubus idaeus L.
   Common name.—Raspberry.
Parentage: Vajolet x wild mountain raspberry plant.
Plant:
   General habit.—Upright canes; very strong vigor; very good roots system; adapted to medium winter chill conditions such as those of Northern Italy; high productivity in autumn.
   Average height (average).—2.00-2.20 m.
   Spread (average).—0.90 m.
   Shape.—Vase.
   Vegetative bud length.—Medium 4.0-6.0 mm.
Primocane:
   Length (average).—2.0-2.20 m.
   Diameter (average) at 1.0 m height.—1.1-1.2 cm.
   Texture.—Smooth.
   Internode length (average) at 1 m height.—Medium to long: 6.8-7.3 cm.
   Number of fruiting laterals per cane (average).—9-10.
   Young shoot colour.—* RHS: 140A.
   Cane colour.—* RHS: 140B.
   Spines.—Present/absent: present. *Colour references made to R.H.S. Colour Chart of The Royal Horticultural Society of London (Edition V)
Spines:
   Shape.—Narrow conical.
   Size (average).—1.00-1.30 mm.
   Density (average) at 1.2 m height.—3-4 per cm²
   Colour.—* RHS 172A.
Foliage:
   Leaves.—
   Shape.—Compound; odd-pinnate and subdivided in 3-5 ovate and serrate small leaflets.
   Predominant profile in cross section.—Flat.
   Rugosity.—Low.
   Complete leaf Length.—32.4 cm. Width:29.3 cm. Number of leaflets: 3-5.
   Upper surface colour.—*RHS: 141B.
   Lower surface colour.—*RHS 137D.
   Lateral leaflets.—Speculate.
   Lateral leaflets average size.—Length:14.1 cm. Width: 9.5 cm.
   Lateral leaflets apex shape.—Pointed.
   Lateral leaflets base shape.—Ovate.
   Lateral leaflets margin type.—Serrate.
   Terminal leaflets average size.—Length: 14.9 cm. Width: 11.0 cm.
   Terminal leaflets apex shape.—Pointed.
   Terminal leaflets base shape.—Slightly cordate.
   Terminal leaflets margin type.—Serrate.
   Petiole.—
   Average length.—9.0 cm.
Flowers:
   Number per plant (average).—213.
   Length (average).—8.87 mm.
   Diameter (average).—27.78 mm.
   Diameter (receptacle).—7.75 mm.
   Shape.—Pentagonal.
   Petal colour.—*RHS: NN155D.
   Fragrance.—Absent.
   Blooming.—July-August.
Fruit:
   Predominant shape.—Conical.
   Weight.—6.2 g.
   Size.—Length: 3.0 cm. width: 2.5 cm.
   Colour of ripe fruit.—* RHS: 40B.
   Colour of immature fruit.—*RHS: 39D.
   Firmness.—Medium (0.04 Kg).
   Drupes per fruit (average).—109.
   Drupe size.—Length: 4.90 mm. width: 4.46 mm. Depth: 6.49 mm.
   Glossiness.—Medium to low.
   Adherence to plug.—Medium.
   Titratable acidity.—8.33 meq/hg.
   Soluble solids.—8.3° Brix.
   Yield.—4.9 kg linear/m (3 plants/m).
   Main bearing type.—On current year's cane in autumn from half August to late October.
   Storage capacity.—Good shelf-life.
Reproductive organs:
   Pistil number per flower (average).—120.
   Pistil size (average).—5.17 mm.
   Stamen number per flower (average).—95.
   Stamen size (average).—5.07 mm.
Seeds:
   Size (average).—Length: 3.90 mm. Width: 2.43 mm.
   Shape.—Kidney-shaped.
Comparison with parental and commercial cultivar:

TABLE 1
Comparison table between variety and parents/other

| Characteristic | EVITA | TITANIA |
|---|---|---|
| fruit color | Orange red | Red |
| fruit size | Medium-large | Very large |
| plant vigor | vigorous | Very vigorous |
| Plant (size/vigour) | high | Very high |

What is claimed:

1. A new and distinct cultivar of raspberry plant as substantially herein shown and described.
